(19) United States
(12) Patent Application Publication
(10) Pub. No.: US 2006/0275777 A1
(43) Pub. Date: Dec. 7, 2006

(54) Novel strategies for protein vaccines

(76) Inventor: Ernst Rudolf Waelti, Muenchenbuchsee (CH)

Correspondence Address: JONES DAY, 555 SOUTH FLOWER STREET FIFTIETH FLOOR, LOS ANGELES, CA, 90071, US

(21) Appl. No.: 11/144,172

(22) Filed: Jun. 3, 2005

(51) Int. Cl. C12Q 1/70; C12Q 1/68

(52) U.S. Cl. 435/6; 435/5

(57) ABSTRACT

A prerequisite for clinical vaccines is the construction of safe and highly immunogenic reagents able to generate efficient immune responses against target antigens. Lipid based delivery vesicles, including virosomes, as clinically approved safe vaccines, can be used to elicit both humoral and cell-mediated responses against protein antigens and mediate effective immune responses against the target pathogen and/or induce tumor rejection. Thus the compositions of the present invention are useful either as a primary vaccination or as a boost in combination with other vaccines in a context of an adjuvant treatment plan.

### FIELD OF THE INVENTION

This invention relates to the fields of immunology and immunotherapy for cancer and infectious diseases.

### BACKGROUND OF THE INVENTION

Various publications or patents are referred to in parentheses throughout this application to describe the state of the art to which the invention pertains. Each of these publications or patents is incorporated by reference herein.

The immune system patrols the tissues of the body and eliminates cancerous or infected cells by a process called immune surveillance. Immune recognition and elimination of abnormal cells, such as virally infected cells and tumor cells, depends on the expression of certain proteins, or antigens, by the abnormal cells which distinguishes them from normal cells. In the case of cancer, proteins that enable the immune system to discriminate between normal and neoplastic cells include those which are expressed only by tumor cells (i.e. tumor-specific antigens, including differentiation antigens, mutated proteins, and proteins of viral origin), as well as those which are present in both normal and tumor cells but overexpressed in tumor cells (also known as tumor-associated antigens). Extensive research in cancer immunology has shown that it is possible to stimulate the patient's own immune cells to recognize and attack the cancer cells. Accordingly, strategies for the therapy of solid and disseminated tumors have been aimed at specifically activating the immune response to tumor cells and at triggering the migratory activity of cytotoxic T cells to infiltrate tumor tissue and destroy it.

One approach that has been explored is the use of peptide vaccines to stimulate cancer-specific immune responses. This strategy requires the identification of antigenic peptides presented by HLA class I and/or class II molecules and involves extensive testing and optimization of MHC restriction and presentation. However, even after antigenic peptide sequences are identified and prepared for use in vaccination, there are obstacles that limit the clinical usefulness of peptide vaccines. Peptide vaccines often turn out to be poorly immunogenic in vivo and, more importantly, their ability to generate T-cell responses depends on the individual's genetic background due to MHC polymorphisms in the population. Thus, for each individual of a different genetic background, a different peptide vaccine has to be designed, necessitating time consuming tests in a clinical setting in which patients may not have much time left. Lastly, peptide sequences that are optimized for cytotoxic T cell responses typically do not stimulate helper T cells or antibody production, which limits their immunological effect as some tumors and viral infections have been shown to require at least a helper T response in addition to a cytotoxic response. Vaccine sources that provide the entire antigenic repertoire to the immune system are thus highly preferable to peptide-based approaches.

One promising strategy involves the use of entire proteins or protein domains for use in vaccination protocols. These protein vaccines have the advantage that a broad range of antigenic peptide sequences can be processed and presented by cells, such as antigen-presenting cells, eliciting T cell responses in patients of all HLA types. Furthermore, a protein vaccine can expose the antigenic protein in a native conformation to the humoral arm of the immune system and thus generate useful antibody responses in addition to T-cell responses. Previous protein-based vaccination approaches have included the delivery of target proteins either in free form or encapsulated in lipid-based preparations. However, the immune responses obtained by vaccination with free protein have not been satisfactory. By contrast, while encapsulation of antigenic proteins has been shown to be effective in stimulating both T- and B-cell immune responses, the process of encapsulating proteins in lipid-based vesicles is laborious and inefficient, resulting in the loss of large quantities of non-encapsulated proteins. A protein-based vaccination strategy that avoids an encapsulation step and the concomitant experimental manipulations, that increases the efficiency of protein delivery and enhances target-specific immune responses would thus be a great advance in the art.

### BRIEF DESCRIPTION OF THE DRAWINGS

FIG. 1—Expression of rNeu_{ECD} on a breast cancer cell line and transfected COS cells. A: Neu+ breast cancer cell line (NF9006), B: COS cells transfected with Vir-DNA-Neu_{ECD}, C: COS cells transfected with empty Vir, D: COS transfected with Vir-DNA. (Magnification A, C and D: 280× and B: 560×). Bar is 50 μm.

FIG. 2—FACS analysis of rNeu-transfected syngeneic IT22 fibroblast cell lines: IT22 were co-transfected with a control neomycin vector and with a vector expressing rNeu (IT22-neu). IT22 and IT22-neu cell lines were analyzed for HER-2/neu cell surface expression by indirect immunostaining using the rNeu-specific mAb (7.16.4) followed by RPE-conjugated goat anti-mouse IgG. For analysis of MHC class I expression, the biotin-anti mouse H-2D^{q}/H2L^{q} (KH117) and for MHC class II the biotin anti-mouse I-A^{q} (KH 116) were used, followed by streptavidin-RPE. The dark shaded area indicates control staining with RPE-conjugated goat anti-mouse IgG or streptavidin-RPE alone.

FIG. 3: A—rNeu-specific cytotoxic T cell (CTL) activity in mice vaccinated with different DNA-based vaccines. Mice were vaccinated/boosted with the indicated vaccines: WT-VV i.p. (solid diamond), rVV-Neu_{ECD} i.p. (solid square), fDNA s.c. (solid circle), fDNA-Neu-_{ECD} s.c. (shaded open square), or Vir-DNA-Neu_{ECD} i.p. (solid triangle). 2-5 weeks after the second vaccination, spleens were removed, and CTL-activity was assessed in a XTT-based assay using IT22 and IT22-neu as target cells. Similar results were seen in four independent experiments. B—Humoral immune response in mice vaccinated with different DNA vectors. Mice were vaccinated and boosted with the indicated vaccines, and sera were collected 6 weeks after the first vaccination. NF9006 cells were incubated with a 1:50 dilution of serum, followed by REP-conjugated MAbs specific for mouse IgG and analyzed for fluorescence by FACScan. The mean and standard error of the mean of each group are shown.

FIG. 4: A—Effect of prophylactic vaccination on time to tumor formation. All mice develop tumors within the same time range after tumor cell injection. FvB/N mice were vaccinated and boosted with rVV-Neu_{ECD} i.p. (●), Vir-p^{NeuECD}enc i.p. (Δ), Vir-p^{NeuECD}mem i.p. (□), Vir-p^{NeuECD}enc/mem i.p. (⋄), free p^{NeuECD}+adjuvant s.c. (▾), or empty Vir i.p. (▪). The time from tumor injection to development of palpable tumors was assessed every 3 days. 9 to 15 mice per group were compared. Statistical analysis using the Mann-Whitney rank test was performed. B—The effect of the indicated vaccines on tumor progression. Tumor volume was measured every 3 days with Vernier calibers. Tumor volume was calculated using the formula (π/6)×(largest diameter)×(smallest diameter)². Shown are the combined results of 3 independent experiments with 5 mice per group. Bars represent SEM. Statistical analysis using the ANOVA rank test was performed.

FIG. 5: A—Neu-specific cytotoxic T cell (CTL) activity in mice vaccinated with different protein-based vaccines. Mice were vaccinated/boosted with the following vaccines: rVV-Neu_{ECD} i.p. (●), Vir-p^{NeuECD}enc i.p. (Δ), Vir-p^{NeuECD}mem i.p. (□), free p^{NeuECD}+CFA s.c. (▾), or empty Vir i.p. (▪). 2-5 weeks after the second vaccination, spleens were removed, and CTL-activity was assessed in a XTT-based assay using IT22 and IT22-neu as target cells. The results of one representative experiment are shown. B—Humoral immune response in mice vaccinated with different protein vectors. IgG ELISA titers in sera (1:25 dilution) from mice after immunization with rVV-NeuCD, different virosomal preparations containing pNeuECD, free pNeuECD, and empty virosomes. Negative control mouse sera showed OD450 nm-values between 0.07 and 0.10. Single points represent the mean values of triplicate determinations. The mean and standard error of the mean of each group is shown.

### DETAILED DESCRIPTION OF THE INVENTION

The present invention is based on the unexpected discovery that proteins linked to the surface of lipid-based delivery vesicles can elicit potent antigen-specific cellular and humoral immune responses. The prevailing dogma in protein vaccine design has heretofore taught that protein antigens require encapsulation in lipid-based delivery vesicles in order to generate cytotoxic T cell (CD8⁺) responses because only encapsulated protein antigens are protected from endosomal degradation and are able to enter the cytosolic MHC I class pathway required for presentation to cytotoxic lymphocytes. By contrast, the present invention demonstrates for the first time that target protein antigens can stimulate not only the production of antibodies, but also effective cytotoxic T cell responses against target cells expressing the antigen of interest (such as a tumor or virally infected cell) when they are linked to the surface of lipid-based vesicles. Thus, the present invention makes possible the generation of targeted T- and B-cell responses against a protein antigen without the need to encapsulate the protein antigen and without requiring de novo synthesis of the antigen. Typically, the process of target antigen encapsulation in lipid-based vesicles results in the loss of a significant percentage of non-encapsulated material, i.e. protein and/or DNA, thus being inefficient and costly, if not wasteful. The present invention represents a significant advance in the art of vaccine design and production by simplifying the process of making protein-based vaccines and vastly increasing their yield and production efficiency. The present invention also makes it possible to direct effective immune responses of both the cellular and humoral arm of the immune system against target protein antigens, without being restricted to any patient's particular MHC haplotype, making one vaccine effective for patients of all genotypes.

Accordingly, the present invention provides protein antigens that are linked to the surface of lipid-based vesicles. for the generation of potent immune responses against abnormal cells expressing the protein antigens, i.e. tumor cells and/or virally infected cells. The protein antigens of the present invention are intended to comprise full-length, naturally occurring, as well as truncated proteins, particularly those with deleted intracellular domains, catalytic domains, or other domains that may execute signaling functions. The production of truncated protein antigens is well known in the art and may be accomplish by the use of suitable primers containing translation stop codons at appropriate sites. Primer design is routinely performed by persons of skill in the art and may entail an analysis of the amino acid or cDNA sequence of the full length protein to identify primer sequences that ensure proper truncation of the protein when translated. Because of the public availability databases of protein sequences and their DNA sequences, as well as their domains, none of these experimental manipulations require more than routine experimentation. Without wishing to be bound by any particular theory, it is preferred that the proteins be of sufficient length so as to allow for MHC I pathway processing by more than one, and preferably several different HLA haplotypes. Thus, in preferred embodiments of the present invention the protein antigens of choice have at least 25 amino acid residues. In preferred embodiments of the present invention, the protein antigen is of sufficient length to be able to fold into its native or near-native secondary and tertiary structure. In preferred embodiments the protein antigens of choice thus have at least 50 amino acid residues. In other preferred embodiments of the present invention, the protein antigens of choice have at least 100 amino acid residues. In yet other preferred embodiments, the protein antigens of choice have more than 200 amino acid residues.

Accordingly, in preferred embodiments, the present invention provides tumor protein antigens, or domains thereof, that are linked to the surface of lipid-based vesicles. By tumor protein antigen is meant any protein antigen expressed by tumor cells that may serve as a target for a cytotoxic T-cell response to the tumor. Such antigens can include proteins that are overexpressed by tumors include, for example, CPSF, EphA3, G250/MN/CAIX, HER-2/neu, Intestinal carboxyl esterase, alpha-fetoprotein, M-CSF, MUC1, p53, PRAME, RAGE-1, RU2AS, Telomerase, WT1, among many others known in the art. In addition, protein antigens that are uniquely expressed by tumors are also suitable targets for the compositions and methods of the present invention. Such antigens include, for example, BAGE-1, GAGE-1 through 8, GnTV, HERV-K-MEL, LAGE-1, MAGE-1 through 12, NY-ESO-1/LAGE-2, SSX-2, TRP2/INT2, SSCA-1 and 2, CA125, CO-029, DUPAN-2, NY-BR-15 and 16, prostate-specific membrane antigen, the CTCL tumor antigens, lung cancer antigens, and others known in the art. (See, for example, Roopa Srinivasan and Jedd D Wolchok, Journal of Translational Medicine 2004; Janeway Immunobiology, Chapter 14, p. 568, 2001; Abbas Cellular and Molecular Immunology, Chapter 17, p. 387, 2000; Boon T, Coulie P G, Van den Eynde B., Immunol Today 1997, 18:267-268; as well as the protein products of the genes identified in publicly accessible databases such as that offered by the National Center of Biotechnology Information, and that of the Journal of the Academy of Cancer Immunology. Tumor antigens available at http://www.cancerimmunity.org/peptidedatabase/tumorspecific.htm, for example, include tumor antigens resulting from mutations, such as α-actinin-4, Bcr-Abl fusion protein, Casp-8, β-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomerase; shared tumor-specific antigens, such as Bage-1, Gage 3,4,5,6,7, GnTV, Herv-K-mel, Lage-1, Mage-A1,2,3,4,6,10,12, Mage-C2, NA-88, Ny-Eso-1/Lage-2, SP17, SSX-2, TRP2-Int2; differentiation antigens, such as CEA, gp100/Pmel17, kallikrein 4, mammaglobin-A, Melan-A/Mart-1, PSA, Trp-1/gp75, Trp-2, tyrosinase; antigens overexpressed in tumors, such as adipophilin, CPSF, EphA3, G250/MN/CAIX, Her-2/neu, intestinal carboxyl esterase, α-fetoprotein, M-CSF, Muc1, p53, PRAME, PSMA, Rage-1, RU2AS, survivin, telomerase, WT1; and numerous other antigens.

The present invention also provides viral protein antigens, or domains thereof, that are linked to the surface of lipid-based vesicles. By viral protein antigen is meant any viral or virally derived protein expressed by infected cells that may serve as a target for a cytotoxic T cell response directed at the infected cells. Such viral antigens may include Hepatitis C core, E1 and 2, NS3-5 proteins, Human Immunodeficiency Virus (HIV) p17, p24, p2p7p1p6, Protease, RT, Integrase, Vif, Vpr, Tat, Rev, Vpu, gp 160 and Nef proteins, the Influenza Virus proteins available at the NCBI Influenza Virus Resource, including avian influenza, and many more viral proteins against which an immune response may be desired. It should be evident that any known or newly identified viral protein can be used in the compositions and methods of the present invention to generate effective immune responses against the viral protein antigen of interest. Furthermore, the present invention contemplates the use of proteins from other pathogenic organisms, including bacteria, fungi, protozoa, and others. Because of its simplicity, the present invention requires only the identification and production of a protein of choice in adequate quantities.

Also contemplated for use with the compositions and methods of the present invention are the heat shock proteins, particularly tumor-derived heat shock proteins. Heat shock protein antigens suitable for the purposes of the present invention include Hsp96, calreticulin, members of the Hsp 90 Hsp70 and Hsp60 families, gp96, Hsp10, 40, 110, among many others well known to those of skill in the art.

Any and all of these protein antigens are potentially useful with the compositions and methods of the present invention to stimulate potent anti-tumor and anti-viral immune responses. In some instances it may be desirable to link mixtures of several different types of protein antigens to the surface of lipid-based delivery vehicles. It should be noted that both the amino acid and nucleotide sequences of any known tumor or viral protein antigens are publicly available, either in databases such as GenBank, SwissProt, or Entrez Protein or similar databases, or in publications available through Medline. A skilled artisan will know how to produce the protein antigen of choice, or one or more suitable domains thereof, by following standard cloning and protein purification procedures known in the art, including those disclosed in Sambrook et al., Molecular Cloning, Cold Spring Harbor Laboratoy (2001), Ausubel et al. (eds.) Current Protocols in Molecular Biology, John Wiley & Sons (2000), and Harlow and Lane, Antibodies, A Laboratory Manual, Cold Spring Harbor Laboratory, (1988), among many others. The sequence of the viral or tumor-associated protein of choice can additionally be compared against other protein sequences to identify unique domains of the viral or tumor-associated protein which are not shared by normal proteins. Suitable programs for these purposes include the Conserved Domain Architecture Retrieval Tool (CDART) or any of the NCBI protein blasts known to the skilled practitioner. Thus, for persons of skill in the art it is a matter of routine to produce the protein antigen of interest, or suitable domain thereof, by means of recombinant DNA technology and/or cell culture techniques.

If produced in situ, the proteins may be purified from appropriate sources, e.g., appropriate vertebrate cells e.g., mammalian cells, for instance cells from human, mouse, bovine or rat. Alternatively, the availability of nucleic acid molecules encoding the proteins of choice enables production of the proteins using in vitro expression methods well known in the art. For example, a cDNA or gene may be cloned into an appropriate in vitro transcription vector, for in vitro transcription, followed by cell-free translation in a suitable cell-free translation system. In vitro transcription and translation systems are commercially available, e.g., from Promega Biotech, Madison, Wis., or BRL, Rockville, Md.

Larger quantities of antigenic proteins may be produced by expression in a suitable prokaryotic or eukaryotic system. For example, part or all of a DNA molecule, such as the portion coding for the domain of interest of the antigenic polypeptide, may be inserted into a plasmid vector adapted for expression in a bacterial cell (such as E. coli) or a yeast cell (such as Saccharomyces cerevisiae). Such vectors comprise the regulatory elements necessary for expression of the DNA in the host cell, positioned in such a manner as to permit expression of the DNA into the host cell. The regulatory elements required for expression include appropriate origins of replication, promoter sequences, transcription initiation sequences and optionally, enhancer or termination sequences. Secretion signals may be used to facilitate purification of the resulting protein. An appropriate secretion coding sequence for the secretion of the peptide is operably linked to the 5′ end of the coding sequence for the protein, and this hybrid nucleic acid molecule is inserted into a plasmid adapted to express the protein in the host cell of choice. Plasmids specifically designed to express and secrete foreign proteins are available from commercial sources. For example, if expression and secretion is desired in E. coli, commonly used plasmids include pTrcPPA (Pharmacia); pPROK-C and pKK233-2 (Clontech); and pNH8a, pNH16a, pcDNAII and pAX (Stratagene), among others. However, the production of the antigenic proteins of the instant invention in eucaryotic cells with similar posttranslation modifications is preferred for the purposes of the present invention. For the purposes of the present invention, eukaryotic expression systems, particularly mammalian systems, are strongly preferred because they will provide the posttranslational modifications that occur naturally in the proteins of choice.

The antigenic proteins produced by in vitro transcription and translation or by gene expression in a recombinant prokaryotic or eukaryotic system may be purified according to methods known in the art. Recombinant proteins can be purified by affinity separation, such as by immunological interaction with antibodies that bind specifically to the recombinant protein or fusion proteins such as His tags. Such methods are commonly used by skilled practitioners.

In preferred embodiments of the present invention, the protein antigens of choice are linked to the surface of lipid-based delivery vesicles. Known lipid-based vesicles include virosomes, IRIVs, liposomes, Iscoms, or proteoliposomes. While virosomes, or IRIVs, are the lipid-based delivery vesicles of choice for purposes of the instant invention, liposomes, Iscoms, proteoliposome and other lipid-based vesicles known to persons of skill may also be suitable for the purposes of the present invention. In preferred embodiments, the present invention provides virosomes or IRIVs as protein antigen carrier systems to elicit immune responses leading to tumor rejection and/or clearance of infections. Thus, the present invention provides compositions comprising tumor or viral protein antigens of choice, which may additionally be truncated to delete intracellular, catalytic, or other signaling domains, linked to the surface of virosomes or IRIVs (immunostimulating reconstituted influenza virosome). These compositions can be administered to a patient bearing a tumor or an infection to generate an effective immune response against the tumor, its metastases, or against the infection.

The linkage of the protein antigens to the lipid-based vesicles can be accomplished by covalent attachment between a residue of the protein and a fatty acid of the vesicle membrane, or it can be achieved by a non-covalent association between the protein and the membrane lipids. Numerous lipids exist for the covalent or non-covalent attachment of proteins to the surface of the lipid-based vesicle. Most of these lipids fall into three major classes of functionality: conjugation through amide bond formation, disulfide or thioether formation, or biotin/streptavidin binding. Amide conjugation requires phospholipids with either amine or carboxyl functional groups for conjugation with proteins containing amine, carboxyl, or hydroxy groups. Carboxyacyl derivatives of phosphatidylethanolamine (PE) can be used to achieve the coupling of proteins to the surface of preformed lipid vesicles. Disulfide/thioether conjugation can be performed with lipids for disulfide (PDP-PE) or thioether (MPB-PE or MCC-PE) conjugation of thio-containing proteins or peptides. The maleimide-containing lipid, MPB-PE, for example, can be used to couple a protein to a lipid-based vesicle, such as a virosome. Biotin/streptavidin binding is another method to link proteins to lipid membranes. Biotinylated lipids can have the biotin attached directly to PE (Biotin PE), or they can further contain a spacer between the biotin and the PE.

The choice of linkage between the protein antigen and the surface of the lipid-based delivery vesicle is a matter of routine for a person of skill in the art, and depends on factors of convenience (such as availability of amino acid residues). For example, palmitoylation allows the covalent attachment of fatty acids to cysteine residues of proteins. Alternatively, phospholipids containing crosslinkers capable of reacting with amino groups or SH groups of the protein can be used as membrane lipids. Inclusion of such membrane lipids allows the covalent attachment of the protein to the membrane. Examples of such lipids are DSPE or DOPE cross-linker-MAL or DSPE-cross-linker-NHS. DSPE (distearoylphosphatidylethanolamine) and DOPE (dioloeoylphosphatidylethanolamine) can easily be modified to contain a crosslinker with either a maleimid group (MAL) or an N-hydroxysuccinimidyl group suitable for covalent attachment to protein residues. Alternatively, phospholipids, such as PE can be coupled to N-succinimidylpyridyl dithiopropionate (SPDP) and crosslinked to the thiolated protein antigen. Similarly, phospholipids can be linked to heterobifunctional crosslinkers such as N-γ-maleimidobutyriloxisuccinimide ester (GMBS) and reacted with the free cysteine groups of the protein antigen. Other functionalized phospholipids suitable for conjugation of proteins to membrane lipids of lipid-based delivery vesicles are well known in the art and many are commercially available.

Alternatively, the proteins may associate non-covalently with the lipid membrane of the delivery vesicles. Such noncovalent associations may, for example, include adsorption.

Thus, in preferred embodiments of the present invention, a tumor protein antigen is linked to the surface of virosomes for the generation of anti-tumor immune responses. The tumor-associated protein HER-2/neu is a representative example of a protein antigen that can be used in the compositions and methods of the present invention to demonstrate its effectiveness. HER-2/neu is a protein antigen currently being evaluated as a target for antitumor immunotherapy. Although HER-2/neu is constitutively expressed at low levels on different normal adult tissues, humoral and cellular immunity have been shown in patients with HER-2/neu overexpressing tumors (Disis M, Knutson K, Schiffman K, Rinn K, McNeel D (2000) Pre-existing immunity to the HER-2/neu oncogenic protein in patients with HER-2/neu overexpressing breast and ovarian cancer. Breast Cancer Res Treat 62:245-252; Disis M, Pupa S, Gralow J, Dittadi R, Menard S, Cheever M (1997) High-titer HER-2/neu protein-specific antibody can be detected in patients with early-stage breast cancer. J Clin Oncol 15:3363-3367). Although this immunity is clearly not sufficient to provide patients with protection against malignant tumors, priming or boosting a preexisting immunity may have therapeutic effects (Bernard H, Salazar L, Schiffman K, Smorlesi A, Schmidt B, Knutson K, Disis M (2002) Vaccination against the HER-2/neu oncogenic protein. Endocr Relat Cancer 9:33-44; Disis M, Gooley T, Rinn K, Davis D, Piepkorn M, Cheever M (2002) Generation of T cell immunity to HER-2/neu protein after active immunization with HER-/neu peptide-based vaccines. J Clin Oncol 20:2624-2632). The development of new vaccines targeting tumor-associated protein antigens, such as HER-2/neu, and designed to generate an immune response capable of rejecting cancer is still needed.

Thus, to illustrate one embodiment of the present invention, the extracellular domain of HER-2/neu protein (pNeuECD) is linked to the surface of virosomes. Truncated forms of the protein antigens can be produced that omit the intracellular, catalytic, or other signaling domain, in order to preclude any undesired signaling function of the protein antigens. Determination of intracellular, catalytic, extracellular, signalling, and other functional or conserved domains is a matter of routine in the art and can be accomplished by any of the freely available programs, including NCBI's Conserved Domain Database which may be used to identify the conserved domains present in a protein query sequence. In some cancer patients humoral and/or cellular immune responses against the extracellular part of HER-2/neu have been detected (Disis M L, Calenoff E, McLaughlin G, Murphy A E, Chen W, Groner B, Jeschke M, Lydon N, McGlynn E, Livingston R B, Moe R, Cheever M A (1994) Existent T-cell and antibody immunity to HER-2/neu protein in patients with breast cancer. Cancer Res 54:16-20). The relevance of the extracellular domain of HER-2/neu as immunogen has been tested in strategies using DNA vaccines (Chen Y, Hu D, Eling D, Robbins J, Kipps T J (1998) DNA Vaccines encoding full-length or truncated neu induce protective immunity against neu-expressing mammary tumors. Cancer Res 58:1965-1971; Rovero S, Amici A, Di Carlo E, Bei R, Nanni P, Quaglino E, Porcedda P, Boggio K, Smorlesi A, Lollini P, Landuzzi L, Colombo M, Giovarelli M, Musiani P, Formi G (2000) DNA vaccination against rat HER-2/neu p185 more effectively inhibits carcinogenesis than transplantable carcinomas in transgenic BALB/c mice. J Immunol 165:5133-5142; Wei W, Shi W, Galy A, Lichlyter A, Hernandez S, Groner B, Heilbrun L, Jones R (1999) Protection against mammary tumor growth by vaccination with full-length, modified human ErbB-2 DNA Int J Cancer 81:748-754). Repeated intramuscular injection of plasmid DNA encoding rNeuECD provided intermediate levels of protection against a challenge with tumor cells in mice. Although complete protection was not observed with plasmid DNA, no striking difference in tumor rejection was obtained when plasmid vectors encoding the full-length rNeu protein (Neu), the rNeu extracellular and transmembrane (NeuTM) domain, or the rNeu extracellular (NeuECD) domain were used.

Virosomes are reconstituted from influenza virus envelopes and use the same cell receptor-mediated endocytosis as their viral counterparts (Hernandez L, Hoffmann L, Wolfsberg T, White J (1996) Virus-cell and cell-cell fusion. Annu Rev Cell Dev Biol 12:627-661). The receptor binding and the membrane fusion activity of influenza virus with endosomes are known to be mediated by the major viral envelope glycoprotein HA (Bungener L, Idema J, Veer W, Huckriede A, Daemen T, Wilschut J (2002) Virosomes in vaccine development: induction of cytotoxic lymphocyte activity with virosome-encapsulated protein antigens. J Liposome Res 12:155-163; Huckriede A, Bungener L, ter Veer W, Holtrop M, Daemen T, Palache A, Wilschut J (2003) Influenza virosomes: combining optimal presentation of hemagglutinin with immunopotentiating activity. Vaccine 21:925-931). Similar to viral vectors the mildly acidic pH in the lumen of endosomes triggers the fusion of virosomal with endosomal membranes and thus the release of encapsulated material such as DNA, RNA, or proteins into the cytosol of APCs. Therefore, exogenous antigens encapsulated in virosomes may access the MHC class I pathway without the need of de novo protein synthesis [11, 12, 14, 34]. Not all virosomes are likely to fuse with endosomal membranes, and therefore a fraction is thought to become available for the MHC class II pathway.

In humans, immunization strategies using peptides or peptide-pulsed dendritic cells have been shown to be effective at priming naive T cells against these peptides and proteins derived from TAAgs; however, these strategies have yet to show clinical efficacy [35]. For HER-2/neu, several immunodominant peptides have been identified, including a CTL epitope, E75 (spanning amino acids 369-377), that led to the development of a peptide-based vaccine for clinical applications [36-38]. One of these peptides (E75) was tested in a clinical setting as vaccine and was able to break tolerance and generate an anti-HER-2/neu CTL response in patients [39, 40]. Whereas these T cells easily recognized peptide-pulsed tumor cells, they failed to recognize and lyse HER-2/neu-expressing tumor cells, raising the question whether peptide-based vaccines may induce peptide-specific but not native protein-specific immune responses. Furthermore, drawbacks of synthetic peptides vaccines are their limited application due to the restriction of HLA-A2.1 epitopes in clinical indication, and their lack of standardized methods to immunize patients.

To avoid the restriction of immunodominant epitopes, as well as to generate durable immunity with putative T-helper epitopes, the present invention provides vaccines using the extracellular domain of HER-2/neu protein (pNeuECD). Immunization of rats with human pNeuECD in CFA did elicit an immune response to the rat HER-2/neu antigen (rNeu) but did not protect against tumor formation of rNeu-expressing tumors [41]. The same lack of antitumor response was seen in mice vaccinated with human pNeuECD using montaide 720 as an immunoadjuvant [42]. Along the same lines, plasmid DNA vaccines encoding for the human extracellular domain of HER-2/neu did induce only a partial or no protection from a challenge with human HER-2-expressing tumors [30, 42].

The present invention demonstrates the potential of virosomes as an improved protein carrier system and immunoadjuvant in cancer vaccines. Commercially available virosomal vaccines (INFLEXAL V, EPAXAL) have been shown to be very efficacious and safe [43, 44]. The potential of virosomes as delivery system has been demonstrated for nucleic acids and peptide-based vaccines, e.g., for malaria [45]. Recent reports also concluded that synthetic peptide vaccines administrated s.c. with virosomes were able to induce a strong CTL immunity [25]. The present invention shows that the immunogenic effect of pNeuECD is significantly increased when the protein antigen is linked to the virosomal membrane. The results of the present invention indicate that virosomes are a highly suitable carrier system for protein antigens.

The present invention also provides for the administration of the tumor-associated or viral protein antigens linked to the virosomal surface in a suitable pharmaceutical formulation. By administration or administering is meant providing one or more protein antigen-containing compositions of the invention as a drug, prodrug, or a drug-metabolite, to an individual in need of treatment or prevention of a malignancy or viral infection. Such a drug which contains one or more of the compositions of the present invention, as the principal or member active ingredient, for use in the treatment or prevention of malignancies and viral infections, can be administered in a wide variety of therapeutic dosage forms in the conventional vehicles for topical, oral, systemic, local, and parenteral administration. Thus, the invention provides compositions for parenteral administration which comprise a solution of the compositions of the present invention dissolved or suspended in an acceptable carrier, preferably an aqueous carrier. A variety of aqueous carriers may be used, e.g., water, buffered water, 0.4% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, among many others. Thus, a typical pharmaceutical composition for intradermal infusion could be made up to contain 250 ml of sterile Ringer's solution, and 100 mg of peptide. Actual methods for preparing parenterally administrable compounds will be known or apparent to those skilled in the art and are described in more detail in for example, Remington: The Science and Practice of Pharmacy (“Remington's Pharmaceutical Sciences”) Gennaro A R ed. 20^{th} edition, 2000: Williams & Wilkins PA, USA, which is incorporated herein by reference.

The route and regimen of administration will vary depending upon the stage or severity of the cancer or viral infection to be treated, and is to be determined by the skilled practitioner. For example, the antigenic proteins linked to the virosomal surface can be administered in such oral dosage forms for example as tablets, capsules (each including timed release and sustained release formulations), pills, powders, granules, elixirs, tinctures, solutions, suspensions, syrups and emulsions, or by injection. Similarly, they may also be administered in intravenous (either by bolus or infusion methods), intraperitoneal, subcutaneous, topical with or without occlusion, or intramuscular form. In preferred embodiments, the antigenic polypeptide-containing compositions are administered intraperitoneally, intradermally or subcutaneously. All of these forms are well known to those of ordinary skill in the pharmaceutical arts. Furthermore, the present invention provides for the optimization of the efficacy of protective immunity by changing the injection regimen. Thus, the route of immunization may further improve virosomal vaccination with the compositions and methods of the present invention. In one embodiment of the present invention, strong tumor rejection may be obtained when antigenic proteins linked to the surface of virosomes are injected i.p.

The daily dose of the antigenic proteins linked to the virosomal surface of the invention may be varied over a range from 0.001 to 1,000 mg per adult per day. For oral administration, the compositions are preferably provided in the form of tables containing from 0.001 to 1,000 mg, preferably 0.001, 0.01, 0.05, 0.1, 0.5, 1.0, 2.5, 10.0, 20.0, 50.0, 100.0 milligrams of active ingredient for the symptomatic adjustment of dosage according to signs and symptoms of the patient in the course of treatment. An effective amount of drug is ordinarily supplied at a dosage level of from about 0.0001 mg/kg to about 50 mg/kg of body weight per day. The range is more particular from about 0.0001 mg/kg to 7 mg/kg of body weight per day.

Advantageously, suitable formulations of the present invention may be administered in a single daily dose, or the total daily dosage may be administered in divided doses for example of two, three, or four times daily. The antigenic proteins linked to the virosomal surface of the present invention may be used to prepare a medicament or agent useful for the treatment of tumors or their metastases, and viral infections. Furthermore, the compounds of the present invention can be administered in intranasal form, or via transdermal routes known to those of ordinary skill in the art. To be administered in the form of a transdermal delivery system, the dosage administration will, of course, be continuous rather than intermittent throughout the dosage regimen.

For treatment and prevention of cancers and/or metastases, the antigenic proteins linked to the virosomal surface of the present invention may be administered in a pharmaceutical composition comprising the active compound in combination with a pharmaceutically acceptable carried adopted for topical administration. Topical pharmaceutical compositions may be, for example, in the form of a solution, cream, ointment, gel, lotion, shampoo, or aerosol formulation adapted for application to the skin. These topical pharmaceutical composition containing the compounds of the present invention ordinarily include about 0.005% to 5% by weight of the active compound in admixture with a pharmaceutically acceptable vehicle.

For the treatment and prevention of tumors and metastases, or viral infections, the compositions of the present invention may be used together with other agents known to be useful in treating such malignancies. For combination treatment with more than one active agent, where the active agents can be administered concurrently, the active agents can be administered concurrently, or they can be administered separately at staggered times.

The dosage regimen utilizing the compositions of the present invention is selected in accordance with a variety of factors, including for example type, species, age, weight, sex and medical condition of the patient, the stage and severity of the malignancy or infection, and the particular compound thereof employed. A physician of ordinary skill can readily determine and prescribe the effective amount of the drug required to prevent, counter, or arrest the progress of the malignancy. Optimal precision in achieving concentration of drug with the range that yields efficacy either without toxicity or with acceptable toxicity requires a regimen based on the kinetics of the drug's availability to target sites. This process involves a consideration of the distribution, equilibrium, and elimination of the drug, and is within the ability of the skilled practitioner.

In the methods of the present invention, the compounds herein described in detail can form the active ingredient and are typically administered in admixture with suitable pharmaceutical diluents or excipients suitably selected with respect to the intended form of administration, that is, oral tablets, capsules, elixirs, syrups, and the like, and consistent with conventional pharmaceutical practices. For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium alginate, carboxymethyl cellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, aga, bentonite, xanthan gum and the like.

The liquid forms may be suitably flavored suspending or dispersing agents such as the synthetic and natural gums, for example, tragacanth, acacia, methyl cellulose and the like. Other dispersing agents which may be employed are glycerin and the like. For parenteral administration, sterile suspensions and solutions are desired. Isotonic preparations which generally contain suitable preservatives are employed when intravenous administration is desired. Topical preparations containing the active drug component can be admixed with a variety of carrier materials well known in the art, such as, for example, alcohols, aloe vera gel, allatoin, glycerine, vitamins A or E oils, mineral oil, PPG2 myristyl propionate, and the like, to form, for example, alcoholic solutions, topical cleansers, cleansing creams, skin gels, skin lotions, and shampoos in cream or gel formulations.

The antigenic proteins linked to the virosomal surface or formulation thereof of the present invention may be coupled to a class of biodegradable polymers useful in achieving controlled release of a drug, for example, polylactic acid, polyepsilon caprolactone, polyhydroxy butyric acid, polyorthoesters, polyacetals, polydihyrdo-pyrans, polycyanoacrylates, and crosslinked or amphipathic block copolymers of hydrogels. The antigenic proteins of the present invention can also be administered in the form of liposome delivery systems, such as small unilamellar vesicles, large unilameller vesicles and multilamellar vesicles. Liposomes can be formed from a variety of compounds, including for example cholesterol, stearylamine, and various phosphatidylcholines.

Initial doses can be followed by booster doses, following immunization protocols standard in the art. The immunostimulatory effect of the compositions and methods of the instant invention can be further increased by combining any of the above-mentioned antigenic proteins linked to the surface of virosomes, with an immune response potentiating compound. Immune response potentiating compounds are classified as either adjuvants or cytokines. Adjuvants may enhance the immunological response by providing a reservoir of antigen (extracellularly or within macrophages), activating macrophages and stimulating specific sets of lymphocytes. Adjuvants of many kinds are well known in the art; specific examples include Freund's, alum, mycobacteria such as BCG and M. Vaccae, quil-saponin mixtures such as QS-21 (SmithKline Beecham), and various oil/water emulsions (e.g. IDEC-AF). Cytokines are also useful in vaccination protocols as a result of lymphocyte stimulatory properties. Many cytokines useful for such purposes will be known to one of ordinary skill in the art, including interleukin-2 (IL-2), IL-12, GM-CSF and many others.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents. The preparations of the invention are administered in effective amounts. An effective amount is that amount of a pharmaceutical preparation that alone, or together with further doses, stimulates the desired response. Generally, doses of immunogens ranging from one nanogram/kilogram to 100 miligrams/kilogram, depending upon the mode of administration, are considered effective. The preferred range is believed to be between 500 nanograms and 500 micrograms per kilogram. The absolute amount will depend upon a variety of factors, including the composition selected for administration, whether the administration is in single or multiple doses, and individual patient parameters including age, physical condition, size, weight, and the stage of the disease. These factors are well known to those of ordinary skill in the art and can be addressed with no more than routine experimentation.

In the case of treating cancer, the desired response is inhibiting the progression of the cancer and/or inducing the regression of the cancer and its metastases. These desired responses can be monitored by routine methods or can be monitored according to diagnostic methods of the invention discussed herein. The present invention is not to be limited in scope by the specific embodiments described herein. Indeed, various modifications of the invention in addition to those described herein will become apparent to those skilled in the art from the foregoing description, as well as from the examples. Such modifications are intended to fall within the scope of the appended claims.

The present invention shows that virosomes can be used as carrier and immunoadjuvant for a truncated pNeuECD protein bound to different virosomal constructs (Vir-pNeuECD). Vir-pNeuECD protects a significant number of mice from tumor formation compared with free pNeuECD+ complete Freund's adjuvant (CFA) and this protection correlates with the induction of cytotoxic and humoral immune responses. The present invention thus provides tumor-associated and viral proteins linked to the surface of virosomes as a new and safe carrier system and adjuvant for cancer vaccines.

Taken together the present invention shows that virosomes are a highly suitable carrier system for the delivery of proteins into the cytosol of APCs and therefore effectively stimulate a cellular and humoral immune response and tumor rejection. Furthermore, the application of truncated proteins avoids patient-specific and HLA-restricted peptide vaccines. This model is providing important pre-clinical data necessary for designing human vaccination trials after primary surgical treatment, either as a primary vaccination or as a boost in combination with other vaccines in a context of an adjuvant treatment plan.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. Unless otherwise specified, general cloning and protein expression and purification procedures, such as those set forth in Sambrook et al., Molecular Cloning, Cold Spring Harbor Laboratoy (2001), Ausubel et al. (eds.) Current Protocols in Molecular Biology, John Wiley & Sons (2000), and Harlow and Lane, Antibodies, A Laboratory Manual, Cold Spring Harbor Laboratory, (1988) are used. One skilled in the art may develop equivalent means or reactants without the exercise of inventive capacity and without departing from the scope of the invention.

It will be understood that many variations can be made in the procedures herein described while still remaining within the bounds of the present invention.

### Example 1

This example shows the production of

Material and Methods

Chemicals

N-Hydroxysuccinimide ester of palmitic acid (NHSP), N-(1-(2,3-dioleyloxy)propyl)-N,N,N-trimethylammonium methyl sulfate (DOTAP), and 3-sn-phosphatidylcholine (PC; Sigma, St Louis, Mo., USA).

Mice

Female virgin MMTV/r-Neu FVB mice (H-2q) transgenic for the rat neu protein (rNeu-TG) and FVB/N mice (H2q) were purchased from Charles River, Germany. Laboratory animal care was in accordance with institutional guidelines.

Cell Lines

IT22 3T3 (H-2q) fibroblasts were cotransfected with a SV2-Neo-SP65 and a pBR322-rNeu plasmid. G418-resistent rNeu+ clones (IT22-neu) were selected for their rNeu expression by indirect immunofluorescence by FACS. The syngeneic rNeu+ (H-2q) breast cancer cell line NF9006 derived from a rNeu-TG mouse and the syngeneic rNeu− (H-2q) breast cancer cell line K635 derived from a c-myc-TG mouse have previously been described [15].

Amplification and Cloning of the Extracellular Part of HER-2/Neu (NeuECD)

The DNA sequence coding for the extracellular part of rat HER-2/neu (corresponding to amino acid 1-655) was amplified by PCR. The following primers were used: 5′-AATTCGCAATGATCATCATGGAGCTG-3′ (SEQ ID NO: 1; 5′ primer) and 5′-GCCAGCCCGGTGACATAA-3′ (SEQ ID NO: 2; 3′ primer) using pSV2neuN [16] as template. The 3′ primer contained a stop codon, so that only the extracellular part of HER-2/neu was expressed. The fragment was cloned into pVAX1 (Invitrogen, Cat: V260-20; Groningen, Netherland), amplified, purified, and used as a vaccine, either as free DNA or packed into virosomes.

Immunohistochemistry

Cytospins of NF9006 and Vir-DNA-NeuECD infected COS cells were incubated with a mouse anti-rat HER-2/neu (7.16.4, Oncogene Science) Ab diluted in PBS and goat serum. After incubation with biotin-conjugated goat antimouse Ab (Dako, Copenhagen, Denmark), reactivities were detected using an avidin-biotin complex (Dako) and Newfuchsin substrate (Sigma) according to manufacturer's instructions.

Expression Plasmid Construction, Protein Expression, and Isolation/Purification of the pNeuECD

cDNA encoding the extracellular part of rat Neu (NeuECD) was ligated into MCS of the pBADHisB expression vector (Invitrogen). For protein expression pBADHisB-NeuECD was grown in Escherichia coli to an OD600 of 0.5, protein expression was induced with L-arabinose at a final concentration of 0.2% for 3 h. Cells were then pelleted and sonicated, and pNeuECD was isolated and purified from the collected lysates by quantitative SDS PAGE with Model 491 Prep Cell (Bio-Rad Laboratories, Glattbrugg, Switzerland) according to the manufacturer's instructions. Purified fractions were collected and analyzed in a Western blot.

Fatty Acylation of the pNeuECD

To attach the antigen (pNeuECD) to the surface of the virosomal lipid bilayer, pNeuECD was covalently coupled to palmitic acid using a fatty acylation reaction [17].

Preparation of DNA Plasmid-Virosome Complexes (Vir-DNA-NeuECD)

Plasmid pVAX1-NeuECD was encapsulated in virosomes as follows. A 1.5-ml solution of plasmid (590 μg) was added to 3 ml of HBS (Hepes, 20 mmol/l; NaCl, 150 mmol/l, pH 7.4) and mixed with 1.5 ml of HBS§containing 2.95-mg DOTAP and then ultrasonicated. Furthermore, influenza virosomes containing 70% DOTAP in the lipid membrane were prepared as described previously [18]. DNA plasmid-virosome complexes were prepared by mixing DOTAP-encapsulated plasmid liposomes (6 ml) with 2.8 ml of DOTAP-virosomes, and subsequently fused by ultrasonication at room temperature. The resulting solution contained 66.2 μg plasmid/ml.

Preparation of Vir-pNeuECD mem

Hemagglutinin (HA) from the A/Singapore/6/86 strain of influenza virus was isolated as previously described [18, 19]. Supernatant containing solubilized HA trimer (3.9 mg/ml) in 0.01 M E12E8 was used for the production of virosomes. PC (112 mg) in chloroform was added to a round-bottomed flask, and the chloroform was evaporated by a rotary evaporator. The supernatant (7.1 ml containing 28 mg HA) and 11 ml of palmitoyl pNeuECD were added to the flask. The PC film was solubilized under gentle shaking. The mixture was briefly treated by ultrasonication and then filtered through a 0.2-μm filter. The detergent of the resulting solution was removed by extraction with sterile Biobeads SM-2 (Bio-Rad, Richmond, Calif., USA). The content of palmitoyl pNeuECD was verified by Western blot (see below).

Preparation of Vir-pNeuECDenc

Vir-pNeuECDenc was prepared as described above with the exception that the antigen pNeuECD was added to the mixture. After formation of virosomes, nonencapsulated material was removed by size exclusion chromatography on a High Load Superdex 200 column (Pharmacia, Uppsala, Sweden). The content of pNeuECD was determined by Western blot as mentioned below.

Western blot analysis The pNeuECD in the different preparations was identified using anti-6×His monoclonal antibody (Clontech Laboratories, Palo Alto, Calif., USA) and sheep antimouse AP-conjugated Ig (Chemicon, Temecula, Calif., USA) as secondary antibody. Content of pNeuECD was estimated using QuantiScan (Biosoft, Cambridge, UK).

Generation and Inactivation of Recombinant Vaccinia Virus Vector, rVV-NeuECD

The domain of NeuECD was first cloned into a vaccinia shuttle vector (generous gift from Dr K. Tsung, San Francisco, Calif., USA) enabling insertion and transcription in the viral genome. The insert was flanked by two viral sequences enabling homologous recombination in the A56R loci (hemagglutinin-nonessential gene) of vaccinia virus (Copenhagen strain). A clonal recombinant virus was obtained after several rounds of plaque isolation (using transient gpt selection [20]) on CV-1 cells (ATCC CCL70). Several separately isolated clones were PCR screened and one positive recombinant was then amplified and concentrated on 36% sucrose cushions. Viral solutions were titered on CV-1 cells. Virus replication was inactivated by a limited treatment with 1 μg/ml psoralen (Trioxsalen; Calbiochem, Cambridge, Mass., USA) for 10 min at room temperature followed by 8 min exposure to 354-nm long-wave UV (Stratalinker; Stratagene, La Jolla, Calif., USA) as described previously [8, 21].

FACS Analysis for Neu Expression on Cell Line and rNeu-Specific Antibody Levels in Serum

Syngeneic fibroblast cells (IT22-neu) were analyzed for their rNeu-expression as previously described [22]. RNeu+ NF9006 cells (0.5 ·106) were used to determine rNeu-specific antibodies in sera of mice after different vaccines. The method used and cell analysis for fluorescence on a FACScan has been previously described [23].

Vaccination Protocols

Vaccination/boost studies were performed in female virgin FVB mice as previously described [15] with the following vaccines: 1×108 pfu recombinant vaccinia virus (rVV) encoding for NeuECD (rVV-NeuECD) i.p., or 1×108 pfu wild type vaccinia virus (WT-VV) i.p., or 20 μg of plasmid DNA pVAX1 (fDNA) i.m. or s.c., or 20 μg of pVAX1 encoding for NeuECD (fDNA-NeuECD) i.m. or s.c., or 20 μg of fDNA-NeuECD encapsulated in virosomes (Vir-DNA-NeuECD) i.p. In other sets of experiments the following vaccines were used: 20 μg of free pNeuECD with 50 μl of CFA adjuvant (N-acetylglycosaminyl-(β1-4)-N-acetylmuramyl-L-alanyl-D-isoglutamine; Gerbu, Gaiberg, Germany) s.c., or 20 μg of pNeuECD encapsulated in virosomes (Vir-pNeuECD enc), or 20 μg of pNeuECD bound to virosome membranes (Vir-pNeuECD mem), or a mixture of the two (Vir-pNeuECD enc/mem), or empty virosomes all injected i.p. Two weeks after boost, mice were challenged in the back by a subcutaneous (s.c.) injection of 0.5×106 rNeu+ tumor cells. Tumor formation and size was assessed every 3 days using a calibrator. The tumor progression was monitored at the challenge site for 8 weeks.

CTL Generation

Spleen cells (at 5×105 cells/well) were restimulated in vitro on irradiated IT22-neu cells seeded at 1.5×105 cells/well in 24-well plates. Spleen cells were cocultured for 5 days, then used for the cytotoxicity assay described below.

XTT-Based Cytotoxicity Assay

Lytic function of restimulated effector cells against IT22-neu and IT22 target cells was evaluated at different effector to target ratios (E/T 100:1-0.3:1) in triplicate samples. After overnight coincubation of effector and target cells, 50 μl of XTT-solution (Roche Diagnostics, Rotkreuz, Switzerland) was added to each well according to the manufacturer's instructions, and absorbance at 490 nm was evaluated on an ELISA reader (Bio-Rad). Percentage specific lysis was calculated for each E/T ratio as follows:
%lysis=OD(target alone)−OD[(target+effectors)−(effectors alone)]OD(target alone)
%Neu-specific killing=%killing of specific targetss(IT22-neu)−%killing of unspecified targets(IT22)

ELISA Analysis of Anti-pNeuECD

Anti-pNeuECD specific antibodies in the sera of mice were determined by an ELISA in flat-bottomed plates coated with pNeuECD in 50-mM carbonate buffer (pH 8.5) overnight. Serum samples were diluted in sample buffer 1:25. As a detecting antibody, horseradish peroxidase-labeled sheep antimouse Ig (Amersham Pharmacia Biotech, UK) was used at a dilution of 1:1,000. The reaction was developed by tetramethylbenzidine (TMB) substrate solution and stopped by the addition of 1-m H2SO4.

Statistical Analysis

All statistical analyses were made using the Mann-Whitney rank test. For all cases, results were considered significant if p values were <0.05.

### Example 2

RNeu Protein is Expressed in Cells Transfected with Plasmid Encapsulated Virosomes

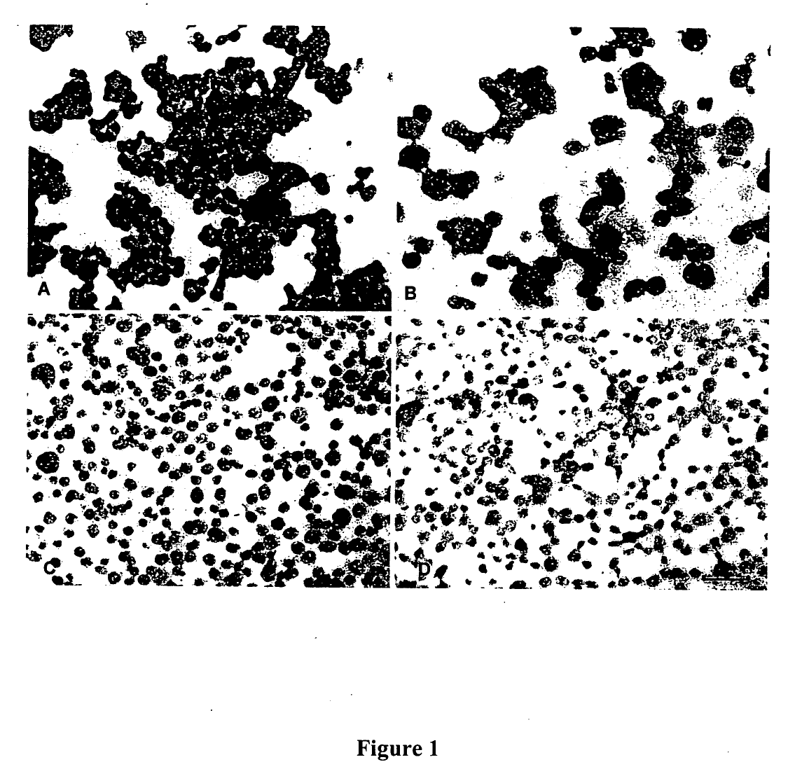

To evaluate whether plasmid DNA (fDNA) encapsulated in virosomes would result in expression of the cloned gene and induce protein production, a plasmid vector was chosen containing a strong promoter (CMV) for optimal expression in mammalian cells and immunostimulatory cytidine-phosphate-guanosine motifs for increased activation of B cells, T cells, and dendritic cells [24]. An fDNA-NeuECD plasmid was engineered, designed to express the extracellular domain of rat Neu (NeuECD). Before testing this plasmid as a vaccine in vivo, the protein expression in transfected COS cells was evaluated in immunohistochemical analysis. Cells transfected with fDNA-NeuECD stained strongly when a mouse antibody (Ab) recognizing an extracellular epitope of rNeu (Ab 7.16.4) was used, whereas no expression could be detected in the same cells when a rabbit antibody recognizing an epitope of the intracytoplasmic part of human/rat Neu was selected (data not shown). To confirm rNeuECD protein production in cells transfected with plasmid DNA-NeuECD encapsulated in virosomes (Vir-DNA-NeuECD), immunohistochemical staining was performed on cytospins of Vir-DNA-NeuECD infected COS cells using again the mouse monoclonal antibody 7.16.4 against rat NeuECD (FIG. 1). The spontaneous breast tumor cell line NF9006, as control, showed a strong staining with mAb 7.16.4 (FIG. 1a). COS cells transfected with Vir-DNA-NeuECD showed a clear staining for NeuECD protein in the cytoplasm of 20-30% of these cells using the same mAb 7.16.4 (FIG. 1b). No staining could be detected in COS cells transfected with empty virosomes or Vir-DNA (no insert) when the same antibody was used (FIG. 1c, d). These results confirmed that cells transfected with plasmid DNA-NeuECD encapsulated in virosomes were capable of producing the antigen.

Prophylactic Vaccination with fDNA-NeuECD Significantly Inhibits Tumor Formation

Virosomes have been used as carriers for the introduction of nucleic acid into mammalian cells in vitro [18]. To evaluate whether Vir-DNA-NeuECD could be used as vaccine and increase rejection of a tumor challenge compared with fDNA-NeuECD, the following experiments were performed: MMTV/r-Neu and/or FVB/N female mice were vaccinated and boosted with recombinant vaccinia virus encoding NeuECD (rVV-NeuECD), with wild-type vaccinia (WT-VV) as negative control, with fDNA (no insert), with fDNA-NeuECD, or with Vir-DNA-NeuECD (as described in “Material and methods”). Two weeks after the boost, mice were challenged with either the syngeneic rNeu+ (NF9006) or rNeu− (K635) breast cancer cell lines and assessed for tumor formation at the challenge site. As shown in Table 1, immunization with fDNA-NeuECD s.c. protected 11 out of 15 mice from tumor formation (tumor incidence 4/15). The specificity of this protection was confirmed, as fDNA-NeuECD vaccination did not protect mice from forming tumors after a challenge with the rNeu-syngeneic breast cancer cell line (K635). In contrast, the immunization with Vir-DNA-NeuECD resulted in a lack of protection, as 13 mice out of 14 developed tumors with NF9006 breast cancer cells. The injection of Vir-DNA-NeuECD s.c. or i.p. showed no difference in tumor rejection.

TABLE 1
Vaccination with fDNA but not with Vir-DNA partially prevents tumor
formation. Mice were vaccinated and boosted with WT-VV i.p.,
rVV-NeuECD i.p., fDNA s.c., fDNA-NeuECD s.c., Vir- DNA-NeuECD
i.p. Two weeks after the boost, each group was challenged s.c. with
either 0.5 · 106 Neu+ tumor cells (NF9006) or 0.5 · 106 Neu− tumor
cells (K635), and tumor progression was monitored at the challenge site
for 8 weeks. The results combine four independent experiments.

| | Tumor incidence | |
|---|---|---|
| | With rNeu⁺ | With rNeu⁻ |
| Vaccination | challenge | challenge |

| | | | | |
|---|---|---|---|---|
| rVV-Neu_{ECD} | 0/13 | (0%) | 5/7 | (71%) |
| WT-VV | 9/9 | (100%) | 6/6 | (100%) |
| fDNA-Neu_{ECD} | 4/15 | (26%) | 5/5 | (100%) |
| fDNA | 11/11 | (100%) | 3/3 | (100%) |
| Vir-DNA-Neu_{ECD} | 13/14 | (92%) | n.d. | |

Table 1-Waccination with fDNA but not with Vir-DNA partially prevents tumor formation.

Mice were vaccinated and boosted with WT-VV i.p., rVV-Neu_{ECD} i.p., fDNA s.c., fDNA-Neu-_{ECD} s.c., Vir-DNA-Neu_{ECD} i.p. Two weeks after the boost, each group was challenged s.c. with either 0.5×10⁶ Neu+ tumor cells (NF9006) or 0.5×10⁶ Neu− tumor cells (K635) and tumor progression was monitored at the challenge site for 8 weeks. The results combine 4 independent experiments.

None of the rVV-NeuECD vaccinated mice developed tumors for an observation period more than 2 months, whereas all mice vaccinated with WT-VV and fDNA (no insert), developed tumors at the challenge site. These results indicated that Vir-DNA-NeuECD seemed incapable of stimulating tumor rejection.

FDNA-NeuECD, but not Vir-DNA-NeuECD, Generates rNeu-Specific Cytotoxic and Humoral Immune Responses

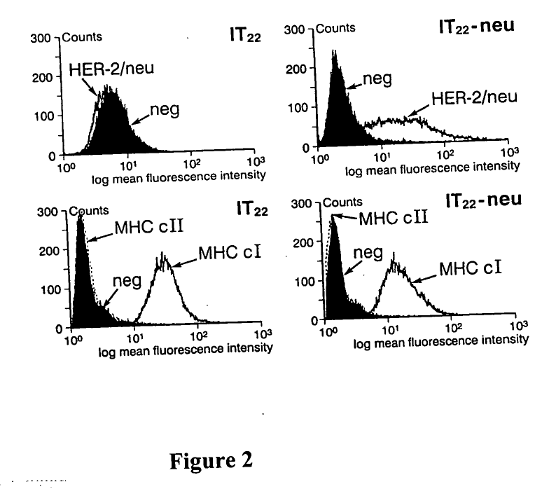

Next, it was examined whether mice vaccinated with fDNA-NeuECD, either free or encapsulated in virosomes (Vir-DNA-NeuECD), would generate rNeu-specific CTLs and/or anti-rNeu Ab responses. A syngeneic rNeu-positive IT22 fibroblast cell line was used as target cells in CTL experiments (see “Material and methods”). IT22-neu expressed significant amounts of cell surface rNeu as monitored by immunofluorescence staining (FIG. 2). No rNeu expression was detected on IT22 fibroblasts. Also shown in FIG. 2 is the MHC class I and MHC class II staining of the two syngeneic fibroblast cell lines. Both cell lines expressed similar levels of MHC class I and were negative for MHC class II.

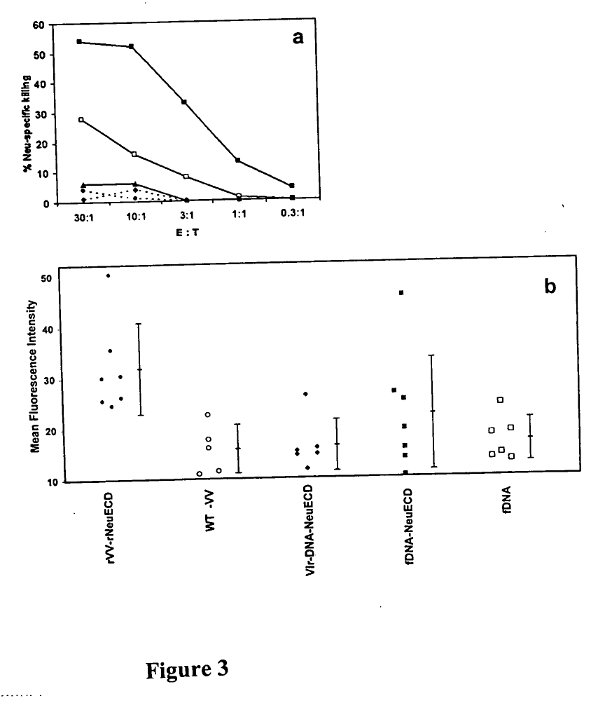

Having demonstrated that vaccination and boost induced tumor rejection in fDNA-NeuECD but not in Vir-DNA-NeuECD vaccinated mice, rNeu-specific CTLs were analyzed in spleen cells of animals vaccinated either with fDNA-NeuECD, Vir-DNA-NeuECD, fDNA (no insert), rVV-NeuECD, or WT-VV, using a colorimetric assay with XTT. As shown in FIG. 3a, the CTL activity was most effective in mice immunized with rVV-NeuECD, with a 6-13-fold greater rNeu-specific lytic activity compared with mice vaccinated with WT-VV. Spleen cells from mice immunized with fDNA-NeuECD also developed rNeuspecific killing in vitro at different effector to target ratios; however, at a reduced percentage level as mice vaccinated with rVV-NeuECD. In contrast, there were only background levels of specific cell lysis in mice vaccinated with Vir-DNA-NeuECD, similar to mice vaccinated with fDNA (no insert) and WT-VV.

To determine whether immunization with the abovementioned vaccines would induce a rNeu-specific humoral immune response, sera of vaccinated and boosted mice were collected at days 49-56 after the first vaccination. The presence of anti-rNeu antibodies was assessed by flow cytometry as previously described [23]. As shown in FIG. 3b, sera from mice injected with rVV-NeuECD showed an impressive level of antibody binding (mean=31.8, SEM=8.9). Similarly, an increase of antibody binding over baseline was noticed in fDNA-NeuECD vaccinated mice (mean=22.3, SEM=11.0). In comparison, mice primed with Vir-DNA-NeuECD produced no rNeu-specific IgG (mean=16.3, SEM=5.0) similar to sera from mice immunized with fDNA or WTVV (both mean=15.8, SEM=4.2).

Taken together, these results indicated that the difference in tumor rejection between fDNA-NeuECD and Vir-DNA-NeuECD correlated with the discrepancy of the induced cellular and humoral immune responses by the two vaccines.

Virosomes can Act as Protein Carrier System and Significantly Increase Tumor Rejection Compared with Free Protein

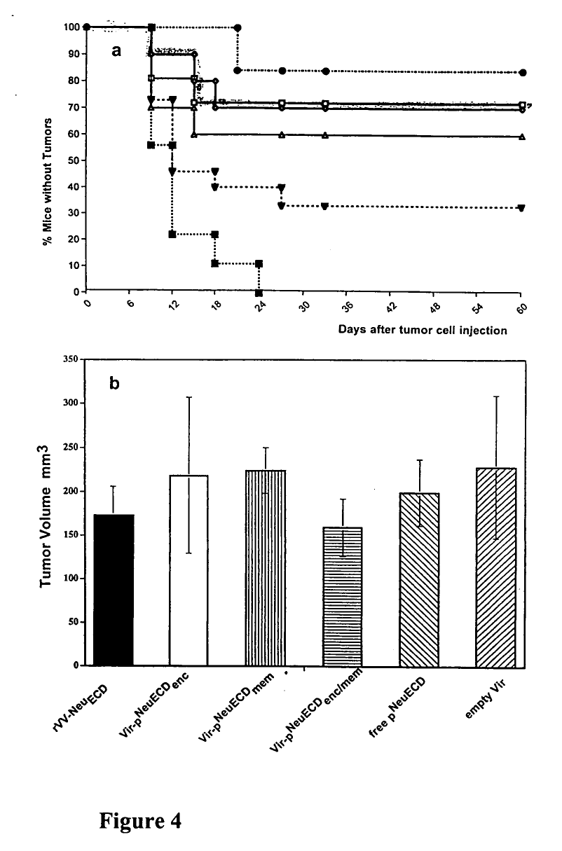

To investigate whether virosomes could be used as carrier and adjuvant for protein TAAg, NeuECD-protein (pNeuECD) was produced using a truncated rNeuECD protein of 90 kDa and two different virosomal constructs were engineered; in the first construct, pNeuECD was encapsulated into the lumen of virosomes (Vir-p NeuECDenc), whereas in the second construct, pNeuECD was inserted into the lipid bilayer by covalently coupling pNeuECD to the palmitoyl fatty acid residues (Vir-pNeuECDmem). In a Western blot analysis the different virosomal constructs demonstrated approximately the same amount of pNeuECD (data not shown). Next, it was investigated whether vaccination with these virosomal constructs (Vir-pNeuECDenc, Vir-pNeuECD mem, or the combination of both Vir-pNeuECDmem/enc) would increase tumor rejection compared to free pNeuECD injected with CFA. Female mice were vaccinated and boosted with pNeuECD+CFA or the different virosomal constructs. These vaccines were tested against rVV-NeuECD and empty Vir as controls. Rejection of a tumor cell challenge was assessed by s.c. injection of rNeu+ and rNeu− syngeneic breast cancer cells. As shown in Table 2, all mice vaccinated with empty Vir developed tumors at the injection site. They all developed their tumors within 24 days after tumor injection (range 8-24 days). Within the observation period of 8 weeks, only 27% (3 out of 11) of Vir-pNeuECD mem-vaccinated mice and only 30% (3 out of 10) of Vir-pNeuECDmem/enc-vaccinated/boosted mice had developed tumors. Mice vaccinated with either of the Vir-pNeuECD constructs developed an impressive protection from tumor formation when compared with mice vaccinated with pNeuECD+CFA, where 10 mice out of 15 tested formed tumors at the injection site. Again, these protections were shown to be rNeu-specific, as mice challenged with rNeu− breast cancer cells were not protected from tumor formation. Mice vaccinated with rVV-NeuECD, Vir-pNeuECDenc, Vir-pNeuECDmem, or Vir-pNeuECDmem/enc showed no significant difference in their time to tumor formation (p<0.5; FIG. 4a). Thus, prophylactic vaccination with pNeuECD in virosomes (Vir-pNeuECDmem/enc, Vir-pNeuECDmem, Vir-pNeuECDenc) significantly prevented the development of tumors at the injection site, in comparison to mice vaccinated with empty Vir as controls (p<0.02, by Mann-Whitney rank test). In contrast, free pNeuECD+CFA showed no significant difference in time to tumor formation compared with empty Vir (p<1.5). As shown in FIG. 4a, all mice developed tumors within 26 days after tumor cell injection, independent of the vaccine used.

TABLE 2
Vaccination with virosome protein prevents tumor formation. Mice were
vaccinated and boosted with rVV-NeuECD i.p., Vir-pNeuECDenc i.p.,
Vir-pNeuECDmem i.p., Vir-pNeuECDenc/mem i.p., emptyVir i.p., or free
pNeuECD. Two weeks after the boost, each group was challenged s.c.
with either 0.5 × 106 Neu+ tumor cells (NF9006) or 0.5 × 106 Neu−
tumor cells (K635), and tumor progression was monitored at the challenge
site for 8 weeks. The results combine three independent experiments.

| | Tumor incidence | |
|---|---|---|
| | With rNeu⁺ | With rNeu⁻ |
| Vaccination | challenge | challenge |

| | | | | |
|---|---|---|---|---|
| rVV-Neu_{ECD} | 2/13 | (15%) | 5/7 | (71%) |
| Vir-p^{NeuECD}enc | 4/10 | (40%) | 5/5 | (100%) |
| Vir-p^{NeuECD}mem | 3/11 | (27%) | 5/5 | (100%) |
| Vir-p^{NeuECD}enc/mem | 3/10 | (30%) | 5/5 | (100%) |
| Empty Vir | 9/9 | (100%) | 3/3 | (100%) |
| Free p^{NeuECD} | 10/15 | (67%) | 5/5 | (100%) |

Table 2-Vaccination with virosome protein prevents tumor formation: Mice were vaccinated

and boosted with rVV-Neu_{ECD} i.p., p^{NeuECD}enc i.p., p^{NeuECD}mem i.p., p^{NeuECD}enc/mem i.p., empty Vir i.p., or free p^{NeuECD}. Two weeks after the boost, each group was challenged s.c. with either 0.5×10⁶ Neu+ tumor cells (NF9006) or o.5×10⁶ Neu− tumor cells (K635) and tumor progression was monitored at the challenge site for 8 weeks. The results combine 3 independent experiments.

Further, the progression of tumor volume in mice with different vaccinations was investigated. As shown in FIG. 4b, there was no significant difference in the tumor volume 14 days after first detection of tumor formation in mice vaccinated with the different Vir-pNeuECD constructs. The tumor progression and tumor volumes were not different between the groups either vaccinated with the different Vir-pNeuECD, rVV-NeuECD (two mice developed tumors), or empty Vir (nine mice developed tumors). These results suggest that Vir-pNeuECD is an effective vaccine for tumor rejection.

Induction of Both CTL and Humoral Immune Response in Vir-pNeuECD Vaccinated Mice

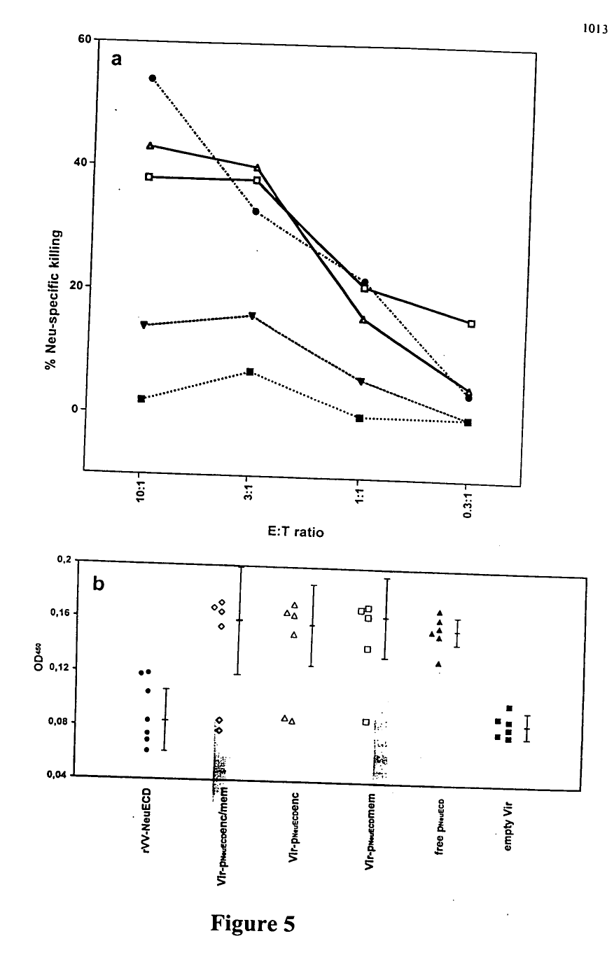

Previous studies have demonstrated that the immunopotentiating effect of modified reconstituted virosomes induced a cellular immune response [14, 25]. To demonstrate whether immunization with different Vir-pNeuECD constructs was capable of inducing rNeuspecific CTL responses, splenocytes were isolated 7 days after booster injection and neu-specific cytotoxic activity was investigated against IT22-neu/IT22 cells. The data depicted in FIG. 5a indicated that mice immunized with Vir-pNeuECDenc or Vir-pNeuECDmem developed equally effective rNeu-specific killing in vitro at different effector to target ratios. Importantly, there was no difference in the CTL activity from that of mice vaccinated with rVV-NeuECD. In contrast, mice immunized with empty Vir showed only background levels of specific cell lysis. Consistent with the lack of tumor rejection, animals vaccinated with free pNeuECD+CFA showed significantly lower CTL activity.

To examine whether pNeuECD, either in the membrane or encapsulated in virosomes, could also induce anti-rNeu Abs, sera of vaccinated and boosted mice were collected 49-56 days after the first vaccination. The presence of anti-rNeu Abs was assessed by flow cytometry [23]. Whereas high levels of rNeu-specific antibodies were detected in sera from mice injected with rVV-NeuECD, no rNeu-specific IgG was detected in sera of Vir-pNeuECD primed mice. Since pNeuECD was expressed and produced in E. coli and therefore unglycosylated, the induced Abs may only recognize the protein backbone. Thus, an ELISA was developed using the same unglycosylated pNeuECD coated to plates as was used in the virosomal vaccine constructs. AntirNeu Abs were now detected in sera of animals immunized with Vir-pNeuECDenc, Vir-pNeuECDmem, or a combination of both and free pNeuECD. In contrast, animals vaccinated with rVV-NeuECD did not develop Abs recognizing the unglycosylated form of pNeuECD in this ELISA. Empty virosomes did not show a rNeuspecific humoral response in either of both systems. Taken together we showed that immunization with Vir-p NeuECD constructs induced a pNeuECD-specific cytotoxic and humoral immune response that correlated with tumor rejection.

### REFERENCES

1. Kapitanovic S, Radosevic S, Kapitanovic M, Ferencic Z, Tavassoli M, Primorac D, Spaventi R (1997) The expression of p185 (HER-2/neu) correlates with the stage of disease and survival in colorectal cancer. Gastroenterology 112:1103-1113
2. Ross J, Sheehan C, Hayner-Buchan A, Ambros R, LKallakury B, Kaufman R, Fisher H, Rifkin M, Muraca P (1997) Prognostic significance of HER-2/neu gene amplification status by fkluorescence in situ hybridization of prostate cancer. Cancer 79:2162-2170
3. Slamon D J, Clark G M, Wong S G, Levin W J, Ullrich A, McGuire W L (1987) Human breast cancer: correlation of relapse and survival with amplification of the HER-2/neu oncogene. Science 235:177-181
4. Tsai C, Chang K, Perng R, Mitsudomi T, Chen M, Gazder A (1993) Correlation of intrinsic chemoresistance of non-smallcell lung cancer cell line with HER-2/neu gene expression bvut not with ras gene mutations. J Natl Cancer Inst 85:897-901
5. Disis M, Knutson K, Schiffman K, Rinn K, McNeel D (2000) Pre-existing immunity to the HER-2/neu oncogenic protein in patients with HER-2/neu overexpressing breast and ovarian cancer. Breast Cancer Res Treat 62:245-252
6. Disis M, Pupa S, Gralow J, Dittadi R, Menard S, Cheever M (1997) High-titer HER-2/neu protein-specific antibody can be detected in patients with early-stage breast cancer. J Clin Oncol 15:3363-3367
7. Moss B (1996) Genetically engineered poxvirus for recombinant gene expression, vaccination and safety. Proc Natl Acad Sci USA 93:11341-11348
8. Zajac P, Schu\ddot{} tz A, Oertli D, Noppen C, Schaefer C, Heberer M, Su\ddot{} pagnoli G, Marti W (1998) Enhanced generation of cytotoxic T lymphocytes using recombinant vaccinia virus expressing human tumor-associated antigens and B7 costimulatory molecules. Cancer Res 58:4567-4571
9. Daemen T, De Haan A, Arkema A, Wilschut J (1998) Liposomes and virosomes as immunoadjuvant and antigencarrier systems in vaccine formulation. Med Appl Liposomes 117-143
10. Hernandez L, Hoffmann L, Wolfsberg T, White J (1996) Virus-cell and cell-cell fusion. Annu Rev Cell Dev Biol 12:627-661
11. Bungener L, Idema J, Veer W, Huckriede A, Daemen T, Wilschut J (2002) Virosomes in vaccine development: induction of cytotoxic lymphocyte activity with virosome-encapsulated protein antigens. J Liposome Res 12:155-163
12. Daemen T, Huckenriede A, Bungener L, Wilschut J (2000) Virosomes as an antigen delivery system. J Liposome Res 10:329-338
13. Peppelenbosch M, DeSmedt M, Pynaert G, Deventer S, Grooten J (2000) Macrophages present pinocytosed exogenous antigen via MHC class I whereas antigen ingested by receptormediated endocytosis is present via MHC class II. J Immunol 165:1984-1991
14. Cusi M G, Zurbriggen R, Valassina M, Bianchi S, Durrer P, Valensin P, Donati M, Glu\ddot{} ck R (2000) Intranasal immunization with mumps virus DNA vaccine delivered by influenza virosomes elicits mucosal and systemic immunity. Virololgy 277:111-118
15. Morrison B W (1994) The genetics of breast cancer. Hematol/Oncol Clin North Am 8:15-28
16. Bargmann C I, Hung M, Weinberg A W (1986) The neu oncogene encodes an epidermal growth factor receptor-related protein. Nature 319:226-230
17. Huang L, Connor J, Wang C (1987) pH-sensitive immunoliposomes. Methods Enzymol 149:88-90
18. Waelti E R, Glu\ddot{} ck R (1998) Delivery to cancer cells of antisense L-myc oligonucleotides incorporated in fusogenic, cationic-lipid-reconstituted influenza-virus envelopes (cationic virosomes). Int J Cancer 77:728-733
19. Skehel J J, Schild G C (1971) The polypeptide composition of influenza-A virus. Virology 44:396-408
20. Falkner F G, Moss B (1988) Escherichia coli gpt gene provides dominant selection for vaccinia virus open reading frame expression vectors. J Virol 62:1849-1854
21. Tsung K, Yim J, Buller MWML, Norton J A (1996) Gene expression and cytopathic effect of vaccinia virus inactivated by psoralen and long-wave UV light. J Virol 70:165-171
22. Céfai D, Favre L, Buri C, Wattendorf E, Marti A, Jaggi R, Gimmi C (2000) The role of Fas ligand expression in promoting escape from immune rejection in an in vivo spontaneous tumor model. Int J Cancer 91:529-537
23. Céfai D, Morrison B, Skell A, Favre L, Balli M, Leunig M, Gimmi C (1999) Targeting HER-2/neu for active-specific immunotherapy in a mouse model of spontaneous breast cancer. Int J Cancer 83:393-400
24. Krieg A S, Yi A, Matson S, Waldschmidt T J, Teasdale B G A R, Koretzky G A, Klinman D M (1995) CpG motifs in bacterial DNA trigger direct B-cell activation. Nature 374:546-549
25. Hunziker I, Zurbriggen R, Glueck R, Engler O, Reichen J, Dai W (2001) Perspectives: towards a peptide-based vaccine against hepatitis C virus. Mol Immunol 38:475-484
26. Bernard H, Salazar L, Schiffman K, Smorlesi A, Schmidt B, Knutson K, Disis M (2002) Vaccination against the HER-2/neu oncogenic protein. Endocr Relat Cancer 9:33-44
27. Disis M, Gooley T, Rinn K, Davis D, Piepkorn M, Cheever M (2002) Generation of T cell immunity to HER-2/neu protein after active immunization with HER-/neu peptide-based vaccines. J Clin Oncol 20:2624-2632
28. Tzahar E, Yarden Y (1998) The ErbB-2/Her2 oncogenic receptor of adenocarcinomas: from orphanhood to multiple stromal ligands. Biochim Biophys Acta 1377:M25-M37
29. Disis M L, Calenoff E, McLaughlin G, Murphy A E, Chen W, Groner B, Jeschke M, Lydon N, McGlynn E, Livingston R B, Moe R, Cheever M A (1994) Existent T-cell and antibody immunity to HER-2/neu protein in patients with breast cancer. Cancer Res 54:16-20
30. Chen Y, Hu D, Eling D, Robbins J, Kipps T J (1998) DNA Vaccines encoding full-length or truncated neu induce protective immunity against neu-expressing mammary tumors. Cancer Res 58:1965-1971
31. Rovero S, Amici A, Di Carlo E, Bei R, Nanni P, Quaglino E, Porcedda P, Boggio K, Smorlesi A, Lollini P, Landuzzi L, Colombo M, Giovarelli M, Musiani P, Forni G (2000) DNA vaccination against rat HER-2/neu p185 more effectively inhibits carcinogenesis than transplantable carcinomas in transgenic BALB/c mice. J Immunol 165:5133-5142
32. Wei W, Shi W, Galy A, Lichlyter A, Hernandez S, Groner B, Heilbrun L, Jones R (1999) Protection against mammary tumor growth by vaccination with full-length, modified human ErbB-2 DNA Int J Cancer 81:748-754
33. Huckriede A, Bungener L, ter Veer W, Holtrop M, Daemen T, Palache A, Wilschut J (2003) Influenza virosomes: combining optimal presentation of hemagglutinin with immunopotentiating activity. Vaccine 21:925-931
34. Correale P, Cusi M, Sabatino M M, Pozzessere L D, Nencini C, Valensin P, Petrioli R, Giorgi G, Zurbriggen R, Gluck R, Francini G (2001) Tumor-associated antigen-specific cytotoxic T cell response in vitro and in a mouse model, induced by TAA-plasmids delivered by influenza virosomes. Eur J Cancer 37:2097-2103
35. Morse M, Clay T, Colling K, Hobeika A, Grabsteiner K, Cheever M, Lyerly H (2003) HER2 dendritic cell vaccines. Clin Breast Cancer Suppl 4:164-172
36. Fisk B, Blevins T L, Wharton J T, Ioannides C G (1995) Identification of an immunodominant peptide of HER-2/neu protooncogene recognized by ovarian tumor-specific cytotoxic T lymphocyte lines. J Exp Med 181:2109-2117
37. Lustgarten J, Theobald M, Labadie C, LaFace D, Peterson P, Disis M, Cheever M (1997) Identification of HER-2/neu CTL epitopes using double transgenic mice expressing HLA-A2 and human CD8. Hum Immunol 52:109-118
38. Yoshino I, Geodegebuure P S, Peoples G E, Parikh A S, DiMaio J M, Lyerly H K, Gazdar A F, Eberlein T J (1994) HER2/neuderived peptides are shared antigens among non-small cell lung cancer and ovarian cancer. Cancer Res 54:3387-3390
39. Disis M L, Gralow J R, Bernhard H, Hand S L, Rubin W D, Cheever M A (1996) Peptide-based, but not whole protein, vaccines elicit immunity to HER-2/neu, an oncogenic selfprotein. J Immunol 156:3115-3158
40. Zaks T, Rosenberg S (1998) Immunization with a peptide epitope (p369-377) from HER-2/neu leads to peptide-specific cytotoxic T lymphocytes that fail to recognize HER-2/neu+ tumors. Cancer Res 58:4902-4908
41. Taylor P, Gerder M, Moros Z, Feldmann M (1996) Humoral and cellular responses raised against the human HER2 oncoprotein are cross-reactive with the homologous product of the neu rpoto-oncogene, but do not protect rats against B 104 tumors expressing mutated neu. Cancer Immunol Immunother 42:179-184
42. Foy T, Bannink J, Sutherland R, McNeill P, Moulton G, Smith J, Cheever M, Grabstein K (2001) Vaccination with HER-2/neu DNA or protein subunits protects against growth of a HER-2/neu expressing murine tumor. Vaccine 19:2598-2606
43. Glueck R, Mischler R, Finkel B, Que J, Scarpa B, Cryz S (1994) Immunogenicity of new virosome influenza vaccine in elderly people. Lancet 344:160-163
44. Holzer B, Hatz C, Schmidt-Sissolak D, Glueck R, Althaus B, Egger M (1996) Immunogenicity and adverse effects of inactivated virosome versus alum-absorbed hepatitis A vaccine: a randomised controlled trial. Vaccine 14:982-986
45. Poeltl-Frank F, Zurbriggen R, Heμga A, Stuart F, Robinson J, Glueck R (1999) Use of reconstituted influenza virus virosomes as an immunopotentiating delivery system for a peptide based vaccine. Clin Exp Immunol 117:496-503

1. An immunostimulatory composition comprising a protein antigen linked to the surface of a lipid-based vesicle.

2. The composition of claim 1, wherein said protein antigen is selected from the group consisting of tumor, viral, heatshock and pathogenic proteins.

3. The composition of claim 1, wherein said protein antigen comprises at least 25 amino acid residues.

4. The composition of claim 1, wherein said protein antigen is covalently linked to the surface of said lipid-based vesicle.

5. The composition of claim 1, wherein said protein antigen is linked to the surface of said lipid-based vesicle by palmitoylation.

6. The composition of claim 1, wherein said protein antigen is noncovalently linked to the surface of said lipid-based vesicle.

7. The composition of claim 1, wherein said protein antigen is truncated.

8. The composition of claim 1, wherein said protein antigen comprises the extracellular domain of Her-2/neu.

9. The composition of claim 1, wherein said lipid-based vesicle is selected from the group consisting of virosomes, IRIVs, liposomes, and iscoms.

10. The composition of claim 8, wherein said lipid based vesicle is a virosome.

11. The composition of claim 9, wherein said protein antigen is covalently linked to the surface of said virosome.

12. The composition of claim 9, wherein said protein antigen is linked to the surface of said virosome by palmitoylation etc.

13. The composition of claim 9, wherein said protein antigen is noncovalently linked to the surface of said virosome.

14. The composition of claim 9, wherein said protein antigen is selected from the group consisting of tumor, viral, heatshock and pathogenic proteins.

15. The composition of claim 9, wherein said protein antigen is truncated.

16. The composition of claim 9, wherein said protein antigen comprises the extracellular domain of Her-2/neu.

17. A method of generating therapeutically effective anti-tumor immune responses comprising administering to a subject the composition of claim 1.

18. A method of generating therapeutically effective anti-viral immune responses comprising administering to a subject the composition of claim 1.


SEQUENCE LISTING (2 sequences)

SEQ ID NO: 1 - DNA, 26 bp, Artificial
  Forward Primer
aattcgcaatgatcatcatggagctg

SEQ ID NO: 2 - DNA, 18 bp, Artificial
  Reverse Primer
gccagcccggtgacataa